(19) United States
(12) Patent Application Publication
(10) Pub. No.: US 2024/0269102 A1
(43) Pub. Date: Aug. 15, 2024

(54) COMPOSITION FOR TOPICAL TREATMENT OF MICROBIAL INFECTIONS

(71) Applicant: HENNEPIN LIFE SCIENCES, LLC, Plymouth, MN (US)

(72) Inventors: Marnie L. Peterson, Jackson, WY (US); Patrick M. Schlievert, Iowa City, IA (US)

(21) Appl. No.: 18/562,917

(22) PCT Filed: May 25, 2022
(86) PCT No.: PCT/US2022/030871

§ 371 Date: Nov. 21, 2023

Related U.S. Application Data
(60) Provisional application No. 63/193,331, filed on May 26, 2021.

(51) Int. Cl. A61K 31/23; A61K 9/06

(52) U.S. Cl. CPC A61K 31/23; A61K 9/06

(57) ABSTRACT

The present disclosure provides compositions and methods for topical treatment of infections. The compositions comprise glycerol monolaurate combined with a required gel, and are administered topically, for example, to treat viral, fungal, bacterial, or protozoan infections.

### CROSS REFERENCE TO RELATED APPLICATION

This application claims the benefit of U.S. Provisional Patent Application Ser. No. 63/193,331, filed May 26, 2021, the disclosure of which is herein incorporated by reference in its entirety.

### BACKGROUND

Some microbial pathogens initiate human illnesses from intact or damaged mucosal and skin surfaces. Many of these pathogens are acquired from other persons or animals, from endogenous sources, or from a myriad of environmental sources. Once in humans, pathogens colonize surfaces primarily as biofilms of organisms, defined as thin-films of organisms attached to host tissues, medical devices, and other microbes through complex networks of polysaccharides, proteins, and nucleic acids. These microbes may also exist as planktonic (broth) cultures in some host tissue environments, such as the bloodstream and mucosal secretions. Similarly, these potential pathogens may exist as either biofilms or planktonic cultures in a myriad of non-living environments.

Glycerol monolaurate (GML) is a naturally occurring glycerol-based compound that has previously been shown to have antimicrobial and anti-inflammatory properties. The present disclosure provides novel, required GML compositions and methods for the treatment of various microbial infections and illnesses resulting from one or more microbial infections. The basis for this disclosure is that GML alone is not effective against one or more microbial infections, but becomes strongly antimicrobial and anti-inflammatory when a new composition, as in this disclosure, is made. Thus, this application is based on composition of matter, requiring GML plus other agents.

### SUMMARY OF THE DISCLOSURE

Simple and well tolerated compositions are needed for applying antimicrobial compounds, such as this novel GML composition, made suitable for human-tolerated use at skin and mucosal surfaces of humans and other vertebrates. The present disclosure addresses this.

In one embodiment, the present disclosure is directed to a composition comprising GML, and a suitable, human-tolerated activator, creating a new required composition. The combination of agents are required for efficacy. One embodiment, the application is use of GML mixed with a suitable non-aqueous-based activating agent. In one embodiment, GML is present in the composition at a concentration from about 50 mg/ml to 350 mg/ml (5% to 35%). The combined GML and activating agent further contains a cellulose derivative, for example either hydroxypropyl cellulose or hydroxyethyl cellulose, or a combination thereof. In a further embodiment, the cellulose derivative is present in the composition up to 5% w/w.

In another embodiment, the present disclosure is directed to a composition comprising GML and a required activating gel. In one embodiment, the composition comprising GML or a derivative thereof and a non-aqueous gel has a pH of about 4.0 to about 6.0 to increase activity. In one embodiment, the required activating gel comprises propylene glycol, ethyl alcohol, diethylene glycol monoethyl ether, polysorbate 20 and 80, polyoxyl 35 castor oil, octyl dodecanol, dimethyl isosorbide, polyethylene glycol 400, and hydroxypropyl cellulose or hydroxyethyl cellulose, or a combination thereof. In one embodiment, hydroxypropyl cellulose and hydroxyethyl cellulose are both present in the composition, each at a concentration of up to 5% w/w.

In one embodiment, the required GML composition provided herein contains propylene glycol at a concentration of about 5-16% (w/w), ethyl alcohol at a concentration of about 4-6% (w/w), diethylene glycol monoethyl ether at a concentration of about 14-16% (w/w), polysorbate 20 at a concentration of about 4-6% (w/w), polysorbate 80 at a concentration of about 4-6% (w/w), polyoxyl 35 castor oil at a concentration of about 4-6% (w/w), octyl dodecanol at a concentration of about 5-15%, dimethyl isosorbide at a concentration of about 5-15% (w/w), polyethylene glycol 400 at a concentration of about 5-15%, and hydroxypropyl cellulose or hydroxyethyl cellulose at a concentration of up to about 5% (w/w).

In one embodiment, the composition contains a cellulose derivative. In a further embodiment, the composition comprises hydroxypropyl cellulose or hydroxyethyl cellulose. In a yet further embodiment, the cellulose is present at a concentration of up to about 5.0% (w/w).

In one embodiment, the pH of the GML composition provided herein is from about 4.0 to about 6.0.

In some embodiments, the composition provided herein contains one or more additional accelerants, in addition to the gel required for antimicrobial activity. In a further embodiment, the accelerant is an organic acid, a chelator, an antibacterial agent, an antifungal agent, an antiviral agent, or a combination thereof. In a further embodiment, the accelerant is a chelator. In even a further embodiment, the accelerant is EDTA.

In another embodiment, the required GML composition provided herein has antimicrobial, antiviral, and/or anti-inflammatory activity. For example, in one embodiment, the composition provided herein is applied topically to humans and other vertebrates, for example for treatment of a bacterial, fungal, or viral infection such as Gardnerella vaginalis, Candida species, and other microbial causes of microbial vaginitis.

Accordingly, in one embodiment, the present disclosure provides required composition of matter and methods for treating microbial infections in a subject in need thereof. In one embodiment, the method comprises topically administering to the subject in need thereof, an effective amount of the required GML composition provided herein. In one embodiment, the composition comprises GML, a required non-aqueous gel for activity-solubilization, and a pharmaceutically acceptable topical carrier.

In one embodiment, the compositions disclosed herein are applied topically with the use of a sponge, wipe, or swab.

In one embodiment, the subject has a bacterial infection. In a further embodiment, the bacterial infection is Staphylococcus (such as Staphylococcus aureus); Streptococcus (such as Streptococcus pneumoniae or Streptococcus agalactiae); Escherichia coli; Gardnerella (such as Gardnerella vaginalis); other potential vaginal pathogens such as Bacteroides species, Atopobium species, Mobiluncus species. Neisseria gonorrhoeae, Chlamydia (such as Chlamydia trachomatis), or Trichomonas vaginalis.

In another embodiment, the subject treated with one of the GML compositions provided herein has a fungal infection. In a further embodiment, the fungal infection is Candida (such as Candida albicans).

In another embodiment, the method of this disclosure involves administering a second active agent selected from the group consisting of antifungal agents, antiviral agents, and antibacterial agents.

### BRIEF DESCRIPTION OF THE TABLE

Table 1 shows the effect of various concentrations of the required GML-Gel composition on the growth of Gardnerella vaginalis and Candida albicans microorganisms. In these experiments, 5 mm diameter sections of porcine vaginal mucosa (PVM; an excellent model for human vaginal mucosa) are incubated for 24 hours (Candida albicans) or 48 hours (Gardnerella vaginalis) in 2 ml of growth medium. Then, 100 or 50 μl of the required GMIL-Gel composition is added and culturing continued for 24 more hours. Counts per milliliter (CFUs/ml) of microbes+standard deviation are then determined from triplicate samples. The starting inoculum of Candida albicans was approximately 104/ml; the starting inoculum for Gardnerella vaginalis as approximately 8.0×10⁵/ml. Because CFUs/ml can be very large, it is customary to refer to them as log₁₀ CFUs/ml. This is what is reported in the Table.

| | | | Log₁₀ CFUs/ml after |
|---|---|---|---|
| | GML (%) + | | 24 hours exposure to |
| Microbe | Required Gel | | Drug or Control (No Drug) |
| Candida albicans | No Drug | | 6.0 ± 0.4 |
| | 5 | (100 μl) | No CFUs* |
| | 5 | (50 μl) | 3.6 ± 0.5* |
| | 15 | (100 μl) | No CFUs* |
| | 15 | (50 μl) | 4.0 ± 0.5* |
| | 25 | (100 μl) | No CFUs* |
| | 25 | (50 μl) | 4.8 ± 0.6* |
| Gardnerella vaginalis | No Drug | | 6.5 ± 0.4 |
| | 5 | (100 μl) | 2.4 ± 0.8* |
| | 5 | (50 μl) | 3.0 ± 0.8* |
| | 15 | (100 μl) | 3.8 ± 0.8* |
| | 15 | (50 μl) | 5.2 ± 0.2* |
| | 20 | (100 μl) | 3.6 ± 0.6* |
| | 20 | (50 μl) | 5.7 ± 0.2* |
| | 25 | (100 μl) | 2.6 ± 0.6* |
| | 25 | (50 μl) | 3.8 ± 0.8* |
| | 35 | (100 μl) | 1.2 ± 0.4* |
| | 35 | (50 μl) | 5.8 ± 0.4 |

Note:
*indicates significantly different from control with p < 0.05 by Student's t test

### DETAILED DESCRIPTION

Glycerol monolaurate (GML) is a naturally occurring glycerol-based compound that has previously been shown to have antimicrobial and anti-inflammatory properties. The present disclosure provides novel, required GML compositions and methods for the treatment of various microbial infections and illnesses resulting from one or more microbial infections. The basis for this disclosure is that GML alone is not effective against one or more microbial infections, but becomes strongly antimicrobial and anti-inflammatory when a new composition, as in this disclosure, is made. Thus, this application is based on composition of matter, requiring GML plus other agents.

The present disclosure provides both topical GML compositions of matter and methods of treatment with the compositions, e.g., by topical administration. The compositions and methods provided, in one embodiment, are used for treating infections topically, for example, by facilitating delivery of effective amounts of the GML composition to a skin or mucosal surface of a subject, e.g., a human. The compositions of the disclosure result in greater patient compliance for topical self-administration due to the less irritating nature of the composition, relative to previously employed topical formulations of antimicrobial compounds.

The term “antimicrobial” means effective in preventing, inhibiting, or arresting the growth or pathogenic effects of a microorganism. “Microorganism” means any bacteria, virus, fungus, or protozoan. In one embodiment, the formulations of the disclosure are used to prevent, inhibit, or arrest the growth of one or more of the following microorganisms: Staphylococcus aureus, Streptococcus (e.g., S. pyogenes, S. agalacticae or S. pneumoniae), Haemophilus influenzae, Pseudomonas aeruginosa, Gardnerella vaginalis, Enterobacteriacae (e.g., Escherichia coli), Bacteroides species, Chlamydia trachomatis, Neisseria gonorrhoeae, Atopobium species, Mobiluncus species, Candida species, Human Immunodeficiency Virus (HIV), Herpes Simplex Virus (HSV), or Trichomonas vaginalis.

“Antibacterial” or “antifungal,” refer to inhibition or arrest of the growth of a bacterium or fungus, a reduction in the severity of or likelihood of developing a bacterial or fungal disease, inducing death of the bacterium or fungus or reduction or inhibition of the pathogenic effects of the respective bacterium or fungus. “Bactericidal” is used interchangeably with “antibacterial.”

“Anti-viral,” refers to inhibition of viral infection or virus replication, a reduction in the likelihood that a subject exposed to a virus will contract the viral disease, or a reduction in the severity of the viral disease.

“Anti-protozoan,” refers to inhibition of protozoan infection or protozoan replication, a reduction in the likelihood that a subject exposed to a virus will contract the protozoan disease, or a reduction in the severity of the protozoan disease.

The term “effective amount,” refers to an amount that is sufficient to cause a beneficial or desired antimicrobial activity, including, without limitation, killing the microorganism or inhibiting microbial infection, growth or toxicity.

The terms “treat,” “treatment,” and “treating” refer to an approach for obtaining beneficial or desired results, for example, clinical results. For the purposes of this disclosure, beneficial or desired results may include inhibiting or suppressing the growth of a microorganism or killing a microorganism; inhibiting one or more processes through which a microorganism infects a cell or subject; inhibiting or ameliorating the disease or condition caused by a microbial infection; or a combination thereof. The terms “treat,” “treatment,” or “treating” also refer to prophylaxis of infection. In some embodiments, the formulations of the disclosure are used to treat vaginal microbial infections.

“Prophylaxis,” can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein, the term “subject” includes humans and other animals. The subject, in one embodiment, is a human.

“Topical,” refers to the application of the composition to any skin or mucosal surface. “Skin surface” refers to the protective outer covering of the body of a vertebrate, generally comprising a layer of epidermal cells and a layer of dermal cells. A “mucosal surface,” refers to a tissue lining of an organ or body cavity that secretes mucous, including but not limited to oral, vaginal, rectal, gastrointestinal, and nasal surfaces. In one embodiment, the formulations of the disclosure are administered topically to the vaginal area.

The term “pharmaceutically acceptable activity enhancer,” refers to a material, diluent, or vehicle that can be applied to skin or mucosal surfaces without undue toxicity, irritation, or allergic reaction. This agent is required for GML activity and thus is part of the composition of matter.

The term “accelerant,” refers to a compound, substance, liquid, powder, or mixture that, when added to the composition, has the effect of enhancing or contributing to the antimicrobial properties of the composition. Accelerants may be an organic acid including, without limitation, lactic acid, ascorbic acid, citric acid, formic acid, benzoic acid, and oxalic acid. The accelerant, in another embodiment, is a chelator, and in one embodiment, is selected from ethylenediaminetetraacetic acid (EDTA), dimercaprol, dimercaptosuccinic acid (DMSA), 2,3-dimercapto-1-propanesulfonic acid (DMPS), alpha lipoic acid (ALA), or combinations thereof. In another embodiment, the accelerant is selected from an antibiotic agent, antifungal agent, antiviral agent, or combination thereof. Antibiotics for use with the disclosure, for example, include aminoglycosides, carbacephems, cephalosporins, glycopeptides, lincosamides, lipopetides, macrolides, monobactams, nitrofurans, penicillins, polypetides, quinolones, sulfuramides, and tetracyclines. Antifungal agents include, without limitation, those of the azole class, polyene class, or echinocanins class, nucleoside analogues, allylamines, griseofulvin, tolnaftate, or selenium compounds. Antiviral agents include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, and zanamivir.

The term “cellulose derivative” refers to any a cellulose-based compound and may include, for example, hydroxyethyl cellulose, hydroxypropyl cellulose, methylcellulose, ethylcellulose, hydroxypropyl methyl cellulose, or cellulose acetate.

The term “biofilm,” means an aggregate of microorganisms, usually bacterial, adhered to one another and growing on a surface. The microbial cells in the biofilm typically produce an extracellular matrix known as an extracellular polymeric substance. Often, this matrix and the density of the aggregate itself significantly increase the antibiotic resistance of the bacteria in the biofilm. Biofilms can be involved in any infections of body surfaces including vaginal mucosae.

In one embodiment, the present disclosure provides a topical composition comprising GML. GML alone has limited antimicrobial activity alone, and this is why the gel is present for required composition of matter. In a further embodiment, the composition comprises the GML plus gel. The gel, in one embodiment, comprises a cellulose derivative. The topical composition provided herein, in one embodiment, comprises a pharmaceutically acceptable topical component of the composition.

In one embodiment, the composition provided herein includes the monoglyceride GML. GML is a fatty acid ester of glycerol, derivative of lauric acid, with the chemical formula C₁₅H₃₀O₄. GML is also known in the art as glyceryl laurate or monolaurin. In this disclosure application GML alone is insufficient for use as an antimicrobial. Thus, the composition of matter requires the use of the non-aqueous gel as an essential component of the active ingredient.

GML can be synthesized in multiple forms including both R and S optical isomers, as well as forms with lauric acid in the 1/3-position and in the 2-position. The composition provided herein, in one embodiment, comprises the R isomer of GML. In another embodiment, the composition provided herein comprises the S isomer of GML. In yet another embodiment, a racemic mixture of isomers is provided in the composition.

Similarly, the topical composition may comprise GML with lauric acid at the 1/3 position, GML with lauric acid at the 2-position, or a combination thereof. R and S isomers of each form, and racemic mixtures thereof, are amenable for use with the present disclosure.

The chemical structure of GML with lauric acid in the 1/3-position is

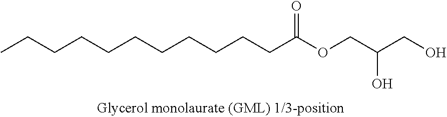 [SMILES: CCCCCCCCCCCC(=O)OCC(O)CO]

The chemical structure of GML with lauric acid in the 2-position is:

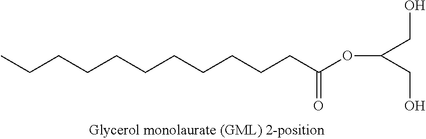 [SMILES: CCCCCCCCCCCC(=O)OC(CO)CO]

The amount of GML in the gel composition can be tailored accordingly to the indication/disease being treated as well as the characteristics of the subject being treated. The amount of GML in the composition may vary depending on, for example, the nature of the infection or illness; the site of administration; the subject's medical history, subject weight, age, sex, and surface area being treated; and whether the subject is receiving any other medications.

In one embodiment, the required gel is present in the topical composition. This may have the composition of: propylene glycol at a concentration of about 5-16% (w/w), ethyl alcohol at a concentration of about 4-6% (w/w), diethylene glycol monoethyl ether at a concentration of about 14-16% (w/w), polysorbate 20 at a concentration of about 4-6% (w/w), polysorbate 80 at a concentration of about 4-6% (w/w), polyoxyl 35 castor oil at a concentration of about 4-6% (w/w), octyl dodecanol at a concentration of about 5-15%, dimethyl isosorbide at a concentration of about 5-15% (w/w), polyethylene glycol 400 at a concentration of about 5-15%, and hydroxypropyl cellulose or hydroxyethyl cellulose at a concentration of up to about 5% (w/w).

In some embodiments, the topical composition comprises one or more accelerants (reduced pH or chelator for example). In a further embodiment, the accelerant is an organic acid, a chelator, an antibacterial agent, an antifungal agent, an antiviral agent, an antiprotozoan, or a combination thereof. In a further embodiment, the accelerant is a chelator. In even a further embodiment, the accelerant is EDTA.

The accelerant, in one embodiment, is EDTA. In a further embodiment, the GML composition provided herein comprises EDTA at a concentration of about 0.00005 M, about 0.0005 M, about 0.005 or about 0.05 M. In another embodiment, a chelator is present in the composition at a concentration of about 0.00005 M to about 0.05 M, about 0.0005 M to about 0.005 M, or about 0.005 to about 0.05 M.

In one embodiment, the pH of the composition is from about 3.5 to about 7.0. In a further embodiment, the pH of the composition is from about 4.0 to about 6.0. In a still further embodiment, the pH of the composition is from about 4.0 to about 4.5.

In one embodiment, the composition is a liquid solution. In another embodiment, the composition is a gel. In another embodiment, the composition is a solid, semi-solid, foam, wax, cream, or lotion.

In one embodiment, the present disclosure provides a method of treating a microbial infection in a subject in need thereof. The microbial infection, in one embodiment, is a bacterial, viral, fungal, or protozoan infection, or a combination thereof.

The GML topical compositions described in this disclosure are less irritating than currently approved antimicrobial compositions, therefore resulting in a more favorable patient compliance rate, as compared to other antimicrobial compositions presently used in the art.

In one embodiment, the method of treating a microbial infection comprises applying an effective amount of one or more of the GML compositions described herein to at least one skin or mucosal surface of a subject.

In some embodiments, the composition is applied to or impregnated in a wipe, sponge, swab, or other material, and then applied to the skin or mucosal surface of the subject using the respective material. the term “swab” refers to a material suitable for applying a liquid, gel, wax, cream, or lotion to a skin or mucosal surface, or the act of applying a liquid, gel, wax, cream, or lotion to the skin or mucosal surface, or the act of collecting a liquid, gel, wax, cream, lotion, or fluid from the skin or mucosal surface. In some embodiments, the material is attached to a holder, for example a stick, wire, rod, or applicator. In further embodiments, the material attached to a holder is attached at one or both ends thereof. In some embodiments, the wipe, sponge, swab, or other material is pre-loaded or packaged together with the composition.

GML plus required gel inhibits microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

Bacteria use two-component signal transduction systems to respond and adapt to environmental changes as well as produce virulence factors. Without wishing to be bound by theory, GML plus required gel is believed to interfere with bacterial signal transduction, either directly or indirectly, through interaction with bacterial plasma membranes. In one embodiment, GML plus required gel bactericidal effect is mediated at least in part by interactions at the bacterial plasma membrane. Similar to GML plus required gel effects on bacterial plasma membranes, the composition has been shown to inactivate certain viruses by disrupting viral lipid envelopes.

In one embodiment, the methods described herein are used to treat a patient with a vaginal microbial infection. In a further embodiment, the vaginal microbial infection is vulvovaginal candidiasis (VVC) or bacterial vaginosis (BV). Women with BV are at risk for pelvic inflammatory disease, endometritis, and vaginal cuff cellulitis, and pregnant women with BV are at further risk of low birth weight, pre-term labor, pre-term delivery, and chorioamnionitis. In patients with VVC or BV, the vaginal flora, which is normally dominated by Lactobacillus species, becomes altered such that other bacterial and/or fungal species dominate. Gardnerella vaginalis and anaerobic bacteria are commonly associated with BV; Candida species, usually C. albicans, are associated with VVC. Accordingly, in one embodiment, the methods provided herein are used to treat a patient with a bacterial infection. In a further embodiment, the infection is a Gardnerella vaginalis or Candida infection.

In some embodiments, the subject to be treated with one or more of the topical compositions provided herein has a viral infection. In a further embodiment, the viral infection is caused by one or more of the following viruses, or class of viruses: influenza virus, herpesviruses (e.g., Herpes Simplex Virus 2), lentiviruses (e.g., Human Immunodeficiency virus).

In some embodiments, the subject to be treated with one or more of the topical compositions provided herein has a fungal or protozoan infection. In a further embodiment, the fungal infection is caused by Candida species (e.g. C. albicans). In a further embodiment, the protozoan infection is caused by Trichomonas vaginalis.

Methods of identifying and diagnosing a bacterial, viral, fungal, or protozoan infection are generally known by those skilled in the art. To assess whether the formulations disclosed herein are useful to treat an infection, methods known to those of ordinary skill in the art may be employed. For example, a BV infection prior to, and after treatment, may be assessed by microscopic examination of vaginal cells.

In one embodiment, a method is provided to remove or kill a biofilm comprising one or more microorganisms. Biofilms can be involved in vaginal infections. In one embodiment, the method comprises administering the topical composition by applying it directly to the biofilm.

1. A composition comprising 5-30% GML in a gel, wherein the GML and gel comprise a biological active composition.

2. The composition of claim 1 comprising 5-30% GML in a non-aqueous gel, wherein the non-aqueous gel comprises propylene glycol, ethyl alcohol, diethylene glycol monoethyl ether, polysorbate 20 and 80, polyoxyl 35 castor oil, octyl dodecanol, dimethyl isosorbide, polyethylene glycol 400, and hydroxypropyl cellulose or hydroxyethyl cellulose, or a combination thereof.

3. The composition of claim 2, wherein both the hydroxypropyl cellulose and hydroxyethyl cellulose are used at a concentration of up to about 5% w/w.

4. The composition of claim 1 further comprising agents that enhance antimicrobial activities.

5. The composition of claim 4 comprising EDTA or having a pH below 7.0
